(19) United States
(12) Patent Application Publication
(10) Pub. No.: US 2002/0022806 A1
(43) Pub. Date: Feb. 21, 2002

(54) Syringe with a plunger

(75) Inventor: Norbert Witowski, Wolfenbuttel (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: SCHERING AKTIENGESELLSCHAFT, Berlin (DE)

(21) Appl. No.: 09/929,081

(22) Filed: Aug. 15, 2001

(30) Foreign Application Priority Data
Aug. 16, 2000 (EP) 00 117 646.0

(51) Int. Cl. A61M 5/315

(52) U.S. Cl. 604/221; 604/230

(57) ABSTRACT

A syringe with a cylinder (1) and a plunger (4) that slides into it is provided, which has a recess (13) in its front side (5) and at least two cylindrical sections (9, 10) on its barrel surface (6), between which a groove (12) reduces the diameter of plunger (4). The pressure that is applied in the syringe acts on outer side (14) of the recess and on an outside face section (15) in a gasket lip (16), which is pressed by this pressure or else by a previously introduced prestressing against the inside cylinder wall and thus seals the syringe contents from the environment.

This invention relates to a syringe with a plunger according to the introductory clause of claim 1. Such syringes are used as prefilled, sterilized syringes, which are provided for the use of injectable diagnostic agents, especially contrast media, which are injected into, for example, blood vessels, organs, organ parts, cavities and other vessels or exert imaging action there.

A syringe according to the introductory clause of claim 1 is known from DE 196 44 622 A1. By the gasket lip that tapers to a point in cross-section, a dimensional stability can be achieved only to an unfavorable extent, so that only a comparatively slight prestressing of the lip can be produced in the radial direction against the cylinder wall. Sealing of the filled syringe in the static state by means of a gasket lip is thus hampered.

The object is thus to offer a syringe, which, in the static state of storage just as in conditions of dynamic loading with production of pressure in the syringe (as in the case of use or autoclaving), ensures an adequate sealing of the contents of the syringe relative to the surrounding area.

The object is achieved by a syringe according to claim 1.

The syringe has in particular a cylinder with a plunger on the inside that closes the cylinder in the proximal direction. The syringe has a plunger which, with its specially shaped gasket lip, advantageously has a stronger seal in the case of dynamic use than in static use relative to the inside wall of the syringe. In addition, the syringe has the advantage that the plunger can be made with a simply shaped face that also makes it possible to bring the recess into the face with little technical expense. The cylindrical sections of the barrel surface improve the guiding of the plunger into the cylinder and prevent tilting.

Preferred embodiments of the invention will emerge from the subclaims.

The embodiment according to claim 3 shows the advantage that in the emptied syringe between the face of the plunger and the identically shaped inner surface of the gasket, a cavity is avoided to a very large extent. As a result, a remaining residual volume in the syringe is minimized.

The embodiment according to claim 4 shows the advantage of a simple shaping in the production of the plunger.

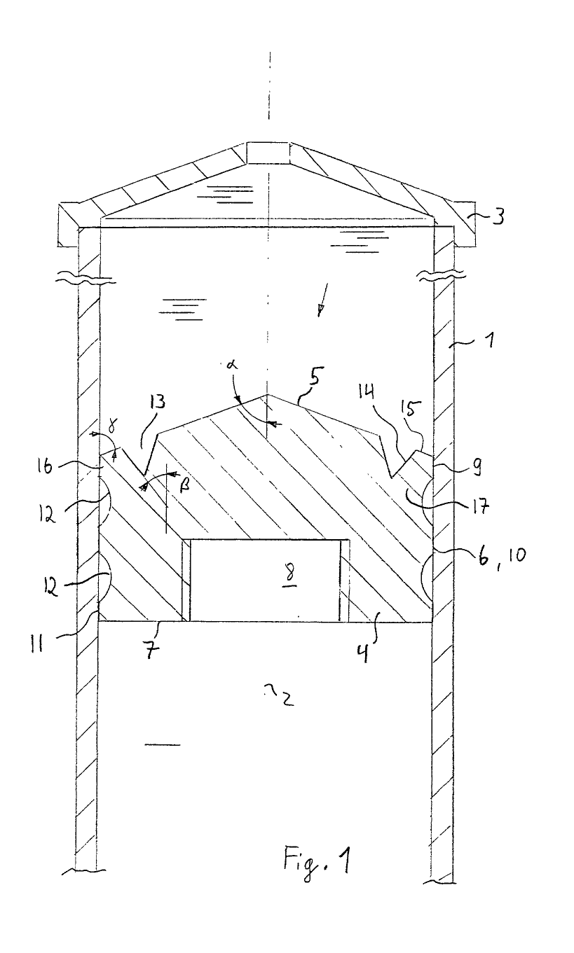

Other practicalities and advantages of the invention will emerge based on the description of an embodiment according to FIG. 1.

FIG. 1 shows a cutout of a cross-sectional view of a first embodiment of the syringe.

As is depicted in FIG. 1, a cylinder 1 is provided with an axis 2. The cylinder preferably consists of glass or plastic. Cylinder 1 is sealed in a known way at its distal end by a gasket 3. A plunger 4 is arranged to move axially in cylinder 1. Axis 2 coincides with the axis of plunger 4.

Plunger 4 has a face 5, a barrel surface 6 and a back area 7. In back area 7, a receptacle 8 is arranged centrally to receive a plunger shaft and said receptacle is threaded. Elastomers are the preferred material of plunger 4. Rubber is an elastomer, which is a vulcanized form of crude rubber. Rubber refers to uncrosslinked, but crosslinkable (vulcanizable) polymers with rubber-elastic properties at room temperature.

Adjoining face 5, barrel surface 6 has a first cylindrical section 9. A second cylindrical section 10 and a third cylindrical section 11 are arranged at intervals, when viewed axially, from first cylindrical section 9 to barrel surface 6. Between first cylindrical section 9 and second cylindrical section 10 and between second cylindrical section 10 and third cylindrical section 11, barrel surface 6 in each case has a circumferential, annular groove 12. The diameter of plunger 4 in the area of groove 12 is therefore smaller than in the first, second or third cylindrical section (9, 10, 11). Groove 12 is designed to be concave in cross-section.

Face 5 is conical around axis 2 and is designed to taper to a point in the distal direction. Between face 5 and axis 2, an angle α is enclosed in the cross-section. Angle α is preferably selected to be greater than 30°. More preferably angle α is between 45° and 90°. In FIG. 1, angle α is equal to 70°.

In face 5 of plunger 4, an annular recess 13 that runs concentrically to axis 2 is provided with a side 14 at a distance from barrel surface 6. Recess 13 is made in the shape of a V. The depth of recess 13 in axial direction is essentially equal to its radial distance from barrel surface 6. Side 14 of recess 11 forms an angle β with its imaginary extension through axis 2. Angle β is preferably less than 90°. More preferably, angle β is between 20° and 60°. In FIG. 1, angle β is equal to 40°.

Through recess 13, an outside face section 15 is formed, which adjoins first cylindrical section 9. Between outside face section 15 and axis 2; an angle γ is enclosed in the cross-section. Angle γ preferably selected as being equal to angle α between face 5 and axis 2 in cross-section. Angle γ is preferably greater than 30°. More preferably, angle γ is between 45° and 90°. In FIG. 1, angle γ is equal to 70°.

Together with outside face section 15, first cylindrical section 19 and groove 12 adjoining first cylindrical section 9, side 14 forms a gasket lip 16. Gasket lip 16 rests above first cylindrical section 9 on the inside wall of cylinder 1. Gasket lip 16 is connected via its base 17 to plunger 4.

After being filled, the syringe is usually stored for a long time. Consequently, in addition to when under dynamic load, even in the static state, plunger 4 must seal the enclosed syringe contents securely relative to the proximal end of cylinder 1. Plunger 4 meets this requirement by the above-described properties in three ways:

By the width of base 17 and the deformability of plunger 4 that is given at least in the area of base 17 and before being inserted into cylinder 1, gasket lip 16 that is connected to base 17 with plunger 4 can be elastically prestressed in the radial direction so that, when inserted, it presses statically against the inside cylinder wall with the initial stressing force that is applied.

By side 14 of recess 13 that is inclined relative to axis 2, a static and a dynamic force component results from the pressure in the interior of the syringe in cylinder 1 in the radial direction to gasket lip 16, which the latter in addition presses against the inside cylinder wall.

By the cylindrical outside shape of first cylindrical section 9, it is ensured that gasket lip 16 is in contact with the inside cylinder wall not just via line contact but rather via a cylinder surface. Possible rough spots in the contact area (and associated leaks) with the inside cylinder wall can be bridged and compensated for by the contact zone that is extended in axial direction.

The shape of gasket lip 16 can be selected so that the force, which occurs in the proximal direction by the friction of gasket lip 16 relative to the inside cylinder wall, and the axially directed force, which produces a tilting moment around base 16 by the pressure in the fluid on outer face section 15 in axial direction, are smaller than the forces in base 17 that stabilize the gasket lip in its shape. As a result, a folding of gasket lip 16 in the proximal direction is prevented.

In another embodiment, recess 13 can be designed with different cross-sectional shapes. Side 14 can thus be designed in bent or corrugated form.

In another embodiment, recess 13 extends with its depth in the radial direction from gasket lip 16 up to axis 2, so that recess 13 assumes the shape of a disk.

In another embodiment, the depth of recess 13 in axial direction is essentially equal to its radial distance from barrel side 6. Based on the shape of recess 13 and the plunger material, other ratios of distance to depth can be selected, for example to optimize the rigidity of plunger 4 in the area of base 17 with adjoining gasket lip 16.

In another embodiment, groove 12 is not concave in cross section but rather stepped with one or more steps. Groove 12 can also be formed by linear diameter reductions or by a combination of the above-mentioned shapes.

In another embodiment, the plunger has a rigid core. As a result, the applied thrusting forces are evenly introduced into the entire plunger.

1. Syringe with a cylinder (1), an axis of symmetry (2), an outlet-side gasket (3) on one end and an inlet-side plunger (4) with a face (5), a barrel (6) and a back area (7) opposite the face,
whereby barrel (6) that adjoins face (5) has a first section (9) of a first diameter and, some distance from it, at least a second section (10) of the first diameter and between them an area of smaller diameter, and a recess (13) is provided in face (5) at a preset distance from the edge,
characterized in that first section (9) is designed as a cylindrical section, and recess (13) on the barrel side has a side area (14) that is inclined from face (5) to axis of symmetry (2).

2. Syringe with a cylinder according to claim 1, wherein recess (13) is annular.

3. Syringe with a cylinder according to claim 2, wherein recess (13) has a V-shaped cross-section.

4. Syringe with a cylinder according to one of claims 1 to 3, wherein face (5) is designed conically in outlet-side gasket (3).

5. Syringe with a cylinder according to one of claims 1 to 4, wherein area (12) between first section (9) and second section (10) is designed to be concave.

6. Syringe with a cylinder according to one of claims 1 to 5, wherein a cylindrical section of the first diameter is provided adjoining back area (7).

7. Syringe with a cylinder according to one of claims 1 to 6, wherein the radial distance of recess (13) from the outside edge of face (5) is approximately equal to its depth.
